(19) United States
(12) Patent Application Publication
(10) Pub. No.: US 2008/0194953 A1
(43) Pub. Date: Aug. 14, 2008

(54) Implantable Microphone Noise Suppression

(75) Inventor: Martin J. Kerber, Innsbruck (AT)

Correspondence Address: BROMBERG & SUNSTEIN LLP, 125 SUMMER STREET, BOSTON, MA, 02110-1618, US

(73) Assignee: MED-EL ELEKTROMEDIZINISCHE GERAETE GMBH, Innsbruck (AT)

(21) Appl. No.: 12/029,051

(22) Filed: Feb. 11, 2008

Related U.S. Application Data
(60) Provisional application No. 60/889,322, filed on Feb. 12, 2007.

(51) Int. Cl. A61F 11/04; A61B 5/0488; A61B 5/0476; A61B 5/00; A61B 8/00

(52) U.S. Cl. 600/437; 600/546; 600/544; 607/57; 600/300

(57) ABSTRACT

A system and method are described for an implantable sensing system. An implantable sensor generates a sensing signal representative of an internal sensing location of a user. A sensing gate, coupled to the sensor and responsive to the sensing signal, has a sensing gate threshold value such that the sensing signal is coupled from the sensing gate to an implanted signal processor when the sensing signal has a magnitude greater than the sensing gate threshold value, and the sensing signal is blocked when the sensing signal has a magnitude less than the sensing gate threshold value.

This application claims priority from U.S. Provisional Patent Application 60/889,322, filed Feb. 12, 2007, the contents of which are hereby incorporated by reference.

### FIELD OF THE INVENTION

The invention relates to prosthetic implants, and specifically to noise suppression of implanted sensors for prosthetic implants.

### BACKGROUND ART

Various medical devices and sensors may usefully be implantable in a user patient. For example, prosthetic systems such as cochlear implant systems may be usefully implanted in persons suffering from partial or complete deafness.

Deafness may be due to total sensorineural hearing loss where the cochlea does not respond to sound waves, and therefore does not generate electrical signals for transmission to the cochleal nerves. An auditory prosthesis may use a suitable stimulation electrode arrangement capable of stimulating the auditory nerves. One current prosthesis design includes an external transmitter and battery, and an internal receiver. The receiver interacts with electrodes that are surgically placed in the cochlea to allow selective stimulation of the cochlear wall (Hochmair et al., U.S. Pat. No. 4,284,856 and 4,357,497, incorporated herein by reference). The electrodes are typically contained in a substantially flexible electrode carrier having sufficient stiffness to be guided into the cochlea in the desired coiled shape (Hochmair-Desoyer et al., Annals of the New York Academy of Sciences 405:173-182 (1991), incorporated herein by reference).

FIG. 1 shows a section view of an ear with a typical cochlear implant system. A normal ear transmits sounds through the outer ear 101 to the eardrum 102, which moves the bones of the middle ear 103, which in turn excites the cochlea 104. The cochlea 104 includes an upper channel scala vestibuli 105 and a lower channel scala tympani 106 which are connected by the cochlear duct 107. In response to received sounds transmitted by the middle ear 103, the fluid-filled scala vestibuli 105 and scala tympani 106 transmit waves, functioning as a transducer to generate electric pulses that are transmitted to the cochlear nerve 108, and ultimately to the brain.

To overcome sensorineural hearing loss, a cochlear implant system produces direct electrical stimulation of the cochlea 104. A typical system may include an external microphone that provides an audio signal input to a signal processing stage (not shown) where various signal processing schemes can be implemented. For example, signal processing approaches that are well-known in the field of cochlear implants include continuous interleaved sampling (CIS) digital signal processing, channel specific sampling sequences (CSSS) digital signal processing (as described in U.S. Pat. No. 6,348,070, incorporated herein by reference), spectral peak (SPEAK) digital signal processing, and compressed analog (CA) signal processing. Typically, the processed signal is then converted into a digital data format, such as a sequence of data frames, for transmission into an implanted receiver processor 109.

Besides getting the processed audio information to the implanted receiver processor 109, existing cochlear implant systems also need to deliver electrical power from outside the body through the skin to satisfy the power requirements of the implanted portion of the system. FIG. 1 shows an arrangement based on inductive coupling through the skin to transfer both the required electrical power and the processed audio information. As shown in FIG. 1, a primary coil 110 (coupled to the external signal processor) is externally placed adjacent to a subcutaneous secondary coil 111 (coupled to the receiver processor 109). This arrangement inductively couples a radio frequency (rf) electrical signal to the receiver processor 109. The receiver processor 109 is able to extract both a power component from the rf signal it receives, and the audio information for the implanted portion of the system.

In addition to extracting the audio information, the receiver processor 109 also performs additional signal processing such as error correction, pulse formation, etc., and produces a stimulation pattern (based on the extracted audio information) that is sent through connecting leads 112 to an implanted electrode carrier 113. Typically, this electrode carrier 113 includes multiple electrodes on its surface that provide selective stimulation of the cochlea 104. The transmission rf signal for primary coil 110 is typically provided by an external behind-the-ear (BTE) module, which may also contain other system components such as the microphone and the external signal processing arrangement.

Besides deafness, various other medical conditions may be usefully addressed with one or more implantable sensors or electrodes. For example, various vocal cord problems such as bilateral vocal cord paralysis, unilateral vocal cord paralysis, voice problems, swallowing problems, aspiration, dysphagia, etc. may be benefit from an implanted pacemaker which includes one or more sensors. U.S. Pat. No. 7,069,082 describes one specific such system and is incorporated herein by reference in its entirety.

Implanted pacemakers with one or more sensors or electrodes may also be useful in the case of obstructive sleep apnea. For example, U.S. Pat. No. 6,361,494, “Electrode And Method For Measuring Muscle Activity In The Pharyngeal Airways,” describes one specific such system and is incorporated herein by reference in its entirety.

Implanted sensors and electrodes are also useful for the treatment of neurological disorders such as for an overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection, and ejaculation. An example of one such system is provided by U.S. Patent Publication 20070282317, “Implantable Microphone For Treatment Of Neurological Disorders,” filed May 18, 2007, and incorporated herein by reference in its entirety.

Newer implanted prosthetic systems are now implanting within the patient many functional components which previously had been provided externally. For example, a sensing microphone and associated signal processing circuitry may be implanted subcutaneously.

### SUMMARY OF THE INVENTION

Embodiments of the present invention are directed to an implantable sensing system. An implantable sensor generates a sensing signal representative of an internal sensing location of a user. A sensing gate is coupled to the sensor and responsive to the sensing signal. The sensing gate has a sensing gate threshold value such that when the sensing signal is greater than the sensing gate threshold, the sensing signal is coupled from the sensing gate to an implanted signal processor, and when the sensing signal is less than the sensing gate threshold, the sensing signal is blocked.

In various embodiments, the sensing gate threshold may be user-controllable and/or software-controllable. For example, the sensing gate may include a sleep mode in which the sensing gate threshold is set to a high value for reducing noise for sleeping.

In specific embodiments, the implantable sensor may be an implantable pressure transducer such as an implantable microphone. In other embodiments the implantable sensor may be an electromyography signal sensor, an electroencephalography signal sensor, or an electroneurography signal sensor. The sensor may be an ultrasound sensor, a temperature sensor, an acceleration sensor, an impedance sensor, a capacitive sensor, or an inductive sensor.

Embodiments also include an implantable prosthesis system including an implantable sensing system according to any of the above. The prosthesis system may be a cochlear implant system or a middle ear implant system. In other embodiments the prosthesis system may be a pacemaker for vocal cord dysfunctions or for obstructive sleep apnea. Or the prosthesis system may be a neurological disorder treatment system such as for a disorder of at least one of overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection and ejaculation.

Embodiments of the present invention also include a method for an implantable sensing system. A sensing signal representative of an internal sensing location of a user is generated. The sensing signal is coupled to a sensing gate having a sensing gate threshold value. The sensing signal is compared to the sensing gate threshold value. When the sensing signal is greater than the sensing gate threshold value, the sensing signal is coupled from the sensing gate to an implanted signal processor. And when the sensing signal is less than the sensing gate threshold value, the sensing signal is blocked.

In various other embodiments, the sensing gate threshold may be user-controllable and/or software-controllable. For example, the sensing gate may include a sleep mode in which the sensing gate threshold is set to a high value for reducing noise for sleeping.

In specific embodiments, the sensing signal may be a pressure signal, for example, from a microphone. Or the sensing signal may be an electromyography signal, an electroencephalography signal, or an electroneurography signal. The sensing signal may be an ultrasound signal, a temperature signal, an acceleration signal, an impedance signal, a capacitive signal, or an inductive signal.

### BRIEF DESCRIPTION OF THE DRAWINGS

FIG. 1 illustrates a section view of an ear connected to a cochlear implant system according to an embodiment of the present invention.

FIG. 2 illustrates a section view of an ear connected to a middle ear implant system according to an embodiment of the present invention.

FIG. 3 shows various functional blocks in one specific embodiment.

FIG. 4 shows a voltage response curve according to one embodiment.

### DETAILED DESCRIPTION OF SPECIFIC EMBODIMENTS

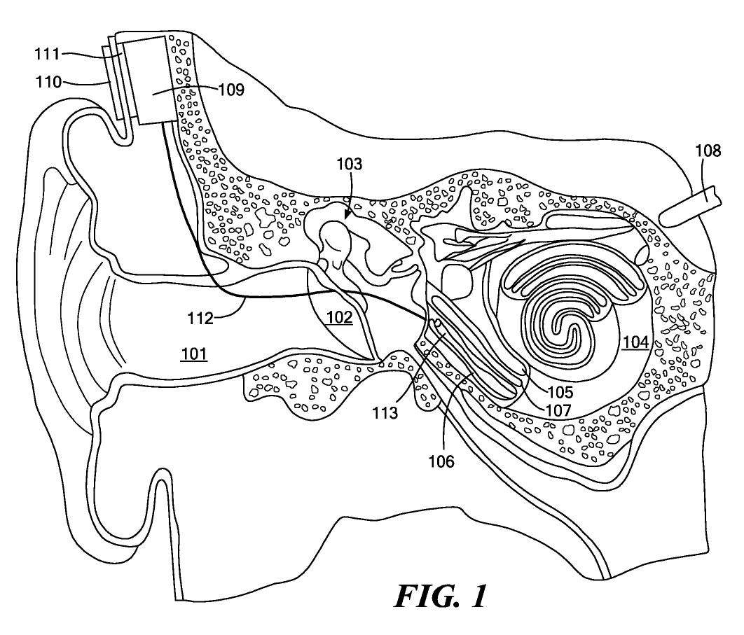

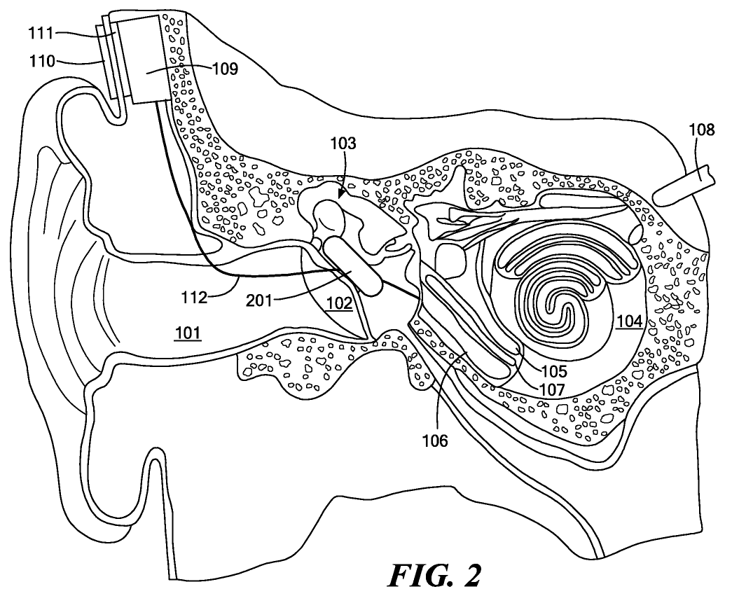

Embodiments of the present invention are directed to reducing noise from an implanted sensor such as a microphone of an implanted prosthetic system such as a cochlear implant system or a middle ear implant system. For example, in the cochlear implant system shown in FIG. 1, the receiver processor 109 may include a sensing microphone which detects acoustic activity across the nearby skin boundary. FIG. 2 illustrates a section view of an ear connected to a middle ear implant system which has a middle ear stimulator 201 instead of an electrode carrier as in a cochlear implant system. The middle ear stimulator 201 mechanically drives the ossicular chain, which in turn stimulates the cochlea 104. A middle ear implant based on a floating mass transducer is described, for example, in U.S. Pat. Nos. 5,913,815; 5,897,486; 5,624,376; 5,554,096; 5,456,654; 5,800,336; 5,857,958; and 6,475,134, each of which is incorporated herein by reference. But again, such a system may have an implanted sensing microphone incorporated into the housing of the receiver processor 109.

One problem with an implanted microphone is that the electric signal it generates can be affected by other noises than simply the acoustic environment near the patient. For example, the microphone produces intrinsic noise (e.g., when the dynamic range is too limited). Also, the body tissue surrounding the microphone housing produces various biological noises. Such undesired and unnecessary noises can be very distracting and otherwise problematic.

In embodiments of the present invention, the microphone output is not passed along to further signal processing circuitry unless and until the amplitude of the microphone signal exceeds a given threshold value. Thus, an implanted sensing microphone generates an electrical microphone signal representative of acoustic activity in an internal sensing location of a user. A sensing gate, coupled to the microphone and responsive to the microphone signal, has a sensing gate threshold value such that the microphone signal is coupled from the sensing gate to an implanted signal processor when the microphone signal has a magnitude greater than the sensing gate threshold, and the microphone signal is blocked when the microphone signal has a magnitude less than the sensing gate threshold.

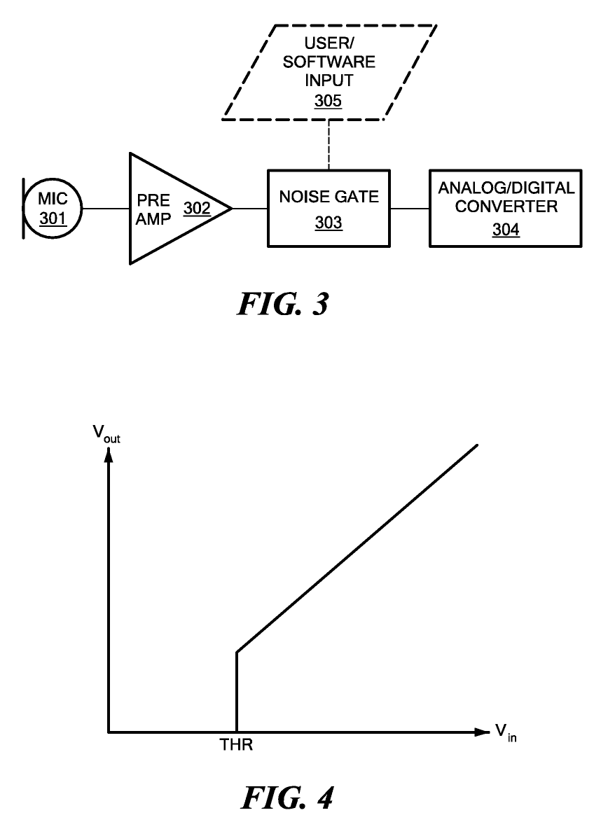

FIG. 3 shows various functional blocks in one specific embodiment. An implanted sensing microphone 301 is located in the housing of an implanted receiver processor 109 just under the skin of the patient user. The sensing microphone senses the nearby acoustic activity and generates a representative electrical microphone signal, which is output to a preamp 302 that linearly amplifies the microphone signal. The preamp 302 couples the amplified microphone signal to the input of sensing gate 303. The sensing gate 303 compares the microphone signal to a sensing gate threshold value. When the microphone signal is less than the threshold, it is blocked by the sensing gate 303. When the microphone signal is greater than the threshold, the sensing gate couples it out the next signal processing stage, in this case, analog/digital converter 304.

In some embodiments, the functionality of the preamp 302 and the sensing gate 303 may be combined such that signals below the threshold are blocked, and those above the threshold are linearly amplified as shown in the voltage response curve of FIG. 4. Some embodiments may also allow the sensing gate threshold to be adjustable, for example, by an optional user/software input 305.

In some embodiments, the sensing gate 303 may be digitally implemented. In that case, the input/output curve of FIG. 4 would be implemented as a lookup table in the signal processing blocks. In such an embodiment, the sensing gate 303 would be located after the analog/digital converter 304 in FIG. 3.

Some embodiments may have a sleep mode that provides a good silence for the user to sleep. In such an embodiment, during the sleep mode the sensing gate threshold is set to a comparably high value in order to suppress most surrounding noises. Optimally, some loud noises such as the sound of an alarm clock or a smoke detector should exceed threshold so as to be heard by the user.

Although the example of cochlear implants is used, other examples of implantable sensors include without limitation implantable pressure transducers such as an implantable microphone, electromyography signal sensors, electroencephalography signal sensors, electroneurography signal sensors, ultrasound sensors, temperature sensors, acceleration sensors, impedance sensors, capacitive sensors, and inductive sensors. Besides cochlear implants, such sensors may be useful, for example, in a middle ear implant system, a pacemaker for vocal cord dysfunctions or for obstructive sleep apnea, or in a neurological disorder treatment system such as for an overactive bladder, urinary urge incontinence, urinary urge-frequency incontinence, urinary urge retention, micturition, fecal incontinence, defecation, peristalsis, pelvic pain, prostatitis, prostatalgia and prostatodynia, erection and ejaculation.

Although various exemplary embodiments of the invention have been disclosed, it should be apparent to those skilled in the art that various changes and modifications can be made which will achieve some of the advantages of the invention without departing from the true scope of the invention.

What is claimed is:

1. An implantable sensing system comprising:
an implantable sensor for generating a sensing signal representative of an internal sensing location of a user; and
a sensing gate coupled to the sensor and responsive to the sensing signal, the sensing gate having a sensing gate threshold value such that:
i. when the sensing signal is greater than the sensing gate threshold, the sensing signal is coupled from the sensing gate to an implanted signal processor, and
ii. when the sensing signal is less than the sensing gate threshold, the sensing signal is blocked.

2. An implantable sensing system according to claim 1, wherein the sensing gate threshold is user-controllable.

3. An implantable sensing system according to claim 1, wherein the sensing gate threshold is software-controllable.

4. An implantable sensing system according to claim 1, wherein the sensing gate includes a sleep mode in which the sensing gate threshold is set to a high value for reducing noise for sleeping.

5. An implantable sensing system according to claim 1, wherein the implantable sensor is a pressure transducer.

6. An implantable sensing system according to claim 5, wherein the implantable pressure transducer is a microphone.

7. An implantable sensing system according to claim 1, wherein the implantable sensor is an electromyography signal sensor.

8. An implantable sensing system according to claim 1, wherein the implantable sensor is one of an electroencephalography signal sensor and an electroneurography signal sensor.

9. An implantable sensing system according to claim 1, wherein the implantable sensor is an ultrasound sensor.

10. An implantable sensing system according to claim 1, wherein the implantable sensor is a temperature sensor.

11. An implantable sensing system according to claim 1, wherein the implantable sensor is an acceleration sensor.

12. An implantable sensing system according to claim 1, wherein the implantable sensor is one of an impedance sensor, a capacitive sensor, and an inductive sensor.

13. An implantable prosthesis system including an implantable sensing system according to any of claims 1-12.

14. A method for an implantable sensing system comprising:
generating a sensing signal representative of an internal sensing location of a user;
coupling the sensing signal to a sensing gate having a sensing gate threshold value;
comparing the sensing signal to the sensing gate threshold value;
when the sensing signal is greater than the sensing gate threshold value, coupling the sensing signal from the sensing gate to an implanted signal processor; and
when the sensing signal is less than the sensing gate threshold value, blocking the sensing signal.

15. A method according to claim 14, wherein the sensing gate threshold is user-controllable.

16. A method according to claim 14, wherein the sensing gate threshold is software-controllable.

17. A method according to claim 14, wherein the sensing gate includes a sleep mode in which the sensing gate threshold is set to a high value for reducing noise for sleeping.

18. A method according to claim 14, wherein the sensing signal is a pressure signal.

19. A method according to claim 18, wherein the pressure signal is a microphone signal.

20. A method according to claim 14, wherein the sensing signal is an electromyography signal.

21. A method according to claim 14, wherein the sensing signal is one of an electroencephalography signal and an electroneurography signal.

22. A method according to claim 14, wherein the sensing signal is an ultrasound signal.

23. A method according to claim 14, wherein the sensing signal is a temperature signal.

24. A method according to claim 14, wherein the sensing signal is an acceleration signal.

25. A method according to claim 14, wherein the sensing signal is one of an impedance signal, a capacitive signal, and an inductive signal.
